(19) United States
(12) Patent Application Publication
(10) Pub. No.: US 2024/0124985 A1
(43) Pub. Date: Apr. 18, 2024

(54) SMALL MODULAR NUCLEAR REACTOR INTEGRATED ENERGY SYSTEMS FOR IN-SITU, ON-DEMAND HYDROGEN GENERATION AND/OR THE PRODUCTION OF SODIUM FORMATE

(71) Applicant: NuScale Power, LLC, Corvallis, OR (US)

(72) Inventors: Francis Y. Tsang, Corvallis, OR (US); José N. Reyes, JR., Corvallis, OR (US); Luis DePavia, Corvallis, OR (US)

(21) Appl. No.: 18/486,971

(22) Filed: Oct. 13, 2023

Related U.S. Application Data
(60) Provisional application No. 63/439,062, filed on Jan. 13, 2023.
(60) Provisional application No. 63/416,327, filed on Oct. 14, 2022.

(51) Int. Cl. C25B 1/042; C02F 1/26; C07C 51/41; C25B 5/00; C25B 15/08

(52) U.S. Cl. CPC C25B 1/042; C02F 1/265; C07C 51/41; C25B 5/00; C25B 15/08; C02F 2103/08

(57) ABSTRACT

Integrated energy systems, such as for use in producing sodium formate and/or processing sodium formate to generate hydrogen as an energy carrier and that produce few or no carbon emissions, and associated devices and methods are described herein. A representative integrated energy system can include a power plant system having multiple modular nuclear reactors. The nuclear reactors can generate electricity and steam for direct use in a sodium formate process or for use in an electrical power conversion system to generate electricity for use in the sodium formate process or for supply to a power grid. Individual ones of the nuclear reactors can be configured to flexibly generate differing outputs of steam or electricity based on a demand state of the power grid—for example, supplying excess electricity and/or steam to the sodium formate process during off-peak hours.

### CROSS-REFERENCE TO RELATED APPLICATIONS

This application claims the benefit of U.S. Provisional Patent Application No. 63/416,327, filed Oct. 14, 2022, and titled “IN-SITU, ON-DEMAND HYDROGEN GENERATION,” and U.S. Provisional Patent Application No. 63/439,062, filed Jan. 13, 2023, and titled “SMALL MODULAR NUCLEAR REACTOR INTEGRATED ENERGY SYSTEMS FOR DESALINATION APPLICATIONS, AND ASSOCIATED DEVICES AND METHODS,” each of which is incorporated herein by reference in its entirety.

### TECHNICAL FIELD

The present technology is directed to Integrated-Energy-Systems (IESs) for the in-situ, on-demand generation of hydrogen, such as from sodium formate (HCOONa), to support an energy imbalance market (EIM) and/or to provide a supplemental backup process to other hydrogen production mechanisms. The present technology is further directed to IESs for producing sodium formate.

### BACKGROUND

Hydrogen is an energy carrier and one of the most important materials to the industrial world. In 2020, roughly 88 million metric tons of hydrogen were produced globally. More than 95% of produced hydrogen is generated through fossil fuels by: (1) steam-methane-reforming (SMR) of natural gas, (2) oxidation of hydrocarbons, (3) coal gasification, and/or (4) by biomass gasification. The foregoing processes generate very large carbon dioxide (CO₂) footprints and have been identified as a major source of greenhouse gases that contribute to climate change and global warming.

Recently, hydrogen production via the electrolysis of water has become an important and integral part of the processes to reduce the greenhouse gas footprint per hydrogen production. Water electrolysis technologies are classified into three basic categories based on the applied electrolyte: (1) high temperature steam electrolysis (HTSE) or solid oxide electrolysis cell (SOEC), (2) liquid alkaline (LA; e.g., alkaline water) electrolysis, and (3) proton exchange membrane (PEM) water electrolysis. Both LA electrolysis and PEM electrolysis are low temperature electrolysis techniques. HTSE and SOEC has the highest hydrogen production efficiencies when input steam temperature is operated in a temperature range of greater than 700° C., and is suitable for constant hydrogen production. LA electrolysis and PEM electrolysis are well-developed technologies that are commercially available and typically operate at much lower temperature and are less efficient than HTSE systems. PEM electrolysis systems have a more compact design than LA electrolysis systems and also have a lower operational input water temperature (typically <100° C.).

HTSE fuel cells are extremely efficient when the input steam temperature is maintained between 700 to 850° C. An HTSE cell can have an all solid-state construction (ceramic and metal) and high operating temperature. The combination of these features leads to several distinctive and attractive attributes including cell and stack design flexibility, multiple fabrication options, and multi-fuel capability choices.

Energy from power generation plants such as, for example, nuclear reactors and/or renewable sources, can be diverted to produce hydrogen via electrolysis of water. Some such systems are described in U.S. patent application Ser. No. 18/116,819, titled “SMALL MODULAR NUCLEAR REACTOR INTEGRATED ENERGY SYSTEMS FOR ENERGY PRODUCTION AND GREEN INDUSTRIAL APPLICATIONS,” and filed Mar. 2, 2023, which is incorporated by reference herein in its entirety. Specifically, the NuScale Power Module™ (NPM) is a 250-megawatt thermal (MWt) integral pressurized water reactor (PWR) that employs gravity-driven natural circulation of the primary coolant for both normal operation and shutdown mode. The NPM, including containment, is fully factory-built and shipped to the plant site by truck, rail, or barge. NuScale's flagship VOYGR-12 power plant design can accommodate up to 12 NPMs, resulting in a total gross output of 924 megawatts electric (MWe). Other configurations include smaller power plant solutions, such as the four-module VOYGR-4 (308 MWe) and the six-module VOYGR-6 (462 MWe).

NuScale VOYGR NPM design, testing, and analysis activities to support a design certification application (DCA) with the U.S. Nuclear Regulatory Commission (NRC) have been underway for several years. Following extensive pre-application activities conducted with the NRC since 2008, the DCA was submitted, and review commenced by the NRC in March 2017. The NRC completed the final phase of the review with the issuance of the Final Safety Evaluation Report (FSER) in August 2020, making NuScale the first ever small modular reactor (SMR) to receive NRC approval.

By 2029, a NuScale power plant will become part of the Carbon Free Power Project (CFPP), an initiative spearheaded by the Utah Associated Municipal Power Systems (UAMPS). UAMPS is a consortium of 48 public power utilities with service areas in eight western states. Interest in NuScale's technology from other power companies continues to build in the United States, as states have or intend to pass legislation for reducing CO₂ emissions and/or establishing clean energy goals.

### BRIEF DESCRIPTION OF THE DRAWINGS

Many aspects of the present technology can be better understood with reference to the following drawings. The components in the drawings are not necessarily to scale. Instead, emphasis is placed on clearly illustrating the principles of the present technology.

FIG. 1 is a partially schematic, partially cross-sectional view of a small modular reactor system configured in accordance with embodiments of the present technology.

FIG. 2 is a partially schematic, partially cross-sectional view of a small modular reactor system configured in accordance with additional embodiments of the present technology.

FIG. 3 is a schematic view of a nuclear power plant system including multiple small modular reactor systems in accordance with embodiments of the present technology.

FIG. 4A is a schematic diagram of an integrated energy system including the power plant system of FIG. 3 in accordance with embodiments of the present technology.

FIGS. 4B and 4C are schematic diagrams of the integrated energy system of FIG. 4A configured during off-peak hours and during peak hours, respectively, in accordance with embodiments of the present technology.

FIG. 5, for example, is a schematic diagram of an integrated energy system including the power plant system of FIG. 3 in accordance with additional embodiments of the present technology.

FIG. 6 is a schematic diagram of a membrane cell for carrying out a chlor-alkali Membrane process via the electrolysis of aqueous sodium chloride in accordance with the prior art.

FIG. 7 is a schematic diagram of a process carried out by a hydrochloric acid production plant for producing hydrochloric acid in accordance with embodiments of the present technology.

FIG. 8A is a schematic diagram of an integrated energy system including the power plant system of FIG. 3 in accordance with additional embodiments of the present technology.

FIGS. 8B and 8C are schematic diagrams of the integrated energy system of FIG. 8A configured during off-peak hours and during peak hours, respectively, in accordance with embodiments of the present technology.

### DETAILED DESCRIPTION

Aspects of the present technology are directed generally toward integrated energy systems, such as for use in producing sodium formate (HCOONa) and/or processing sodium formate to generate hydrogen as an energy carrier. The integrated energy systems of the present technology can have few or nor carbon emissions. In some embodiments, an integrated energy system includes a power plant system having multiple small modular nuclear reactors (SMRs) specifically configured to operate in unison to support a power grid and one or more processes to produce sodium formate and/or process sodium formate (“sodium formate processes”). SMRs are nuclear reactors that are smaller in terms of size (e.g., dimensions) and power compared to large, conventional nuclear reactors. Moreover, they are modular in that some or all of their systems and components can be factory-assembled and transported as a unit to a location for installation. In some aspects of the present technology, the multiple SMRs of the integrated energy system can flexibly and dynamically provide electricity, steam, or a combination of both electricity and steam to the power grid and the sodium formate processes due to the modularity and flexibility of the SMRs. That is, a configuration of the SMRs can be switched during operation to provide varying levels of steam output and electricity output depending on the operational states and/or demands of the power grid and/or sodium formate processes.

In some embodiments, an integrated energy system comprises a sodium formate production system and a power grid both operably coupled to a power plant system comprising multiple SMRs and an electrical power conversion system. Individual ones of the SMRs can heat a coolant into steam that can be (i) directly routed to the sodium formate production system or (ii) routed to the electrical power conversion system for generating electricity. The electricity can be supplied to the power grid, and the sodium formate production system is configured to utilize the steam and/or electricity to produce sodium formate.

More specifically, for example, the sodium formate production system can comprise a desalination plant operably coupled to the power plant system, a brine processing plant operably coupled to the desalination plant, and a sodium formate production plant operably coupled to the brine processing plant and the power plant system. The desalination plant can be positioned to receive seawater, e.g., located at or near a coastal site or brackish water, and steam and/or electricity from the power plant system and use the steam and/or electricity to process the seawater or brackish water to produce brine and clean water. The brine processing plant (e.g., a Chlor-Alkaline Membrane process system) can receive the brine from the desalination plant and process the brine to produce sodium hydroxide solution, hydrogen, and chlorine. The sodium formate production plant can receive the sodium hydroxide from the brine processing plant, carbon monoxide from a carbon monoxide source, and steam and/or electricity from the power plant system and use the steam and/or electricity to process the sodium hydroxide and the carbon monoxide to produce sodium formate. Some aspects of the present technology can reduce or even eliminate the environmental impacts of brine from desalination plants and reprocess some or all of the material components for the production of hydrogen as an energy carrier as well as sodium formate salts and/or hydrochloric acid for other industrial applications.

During operation of the integrated energy system, the power grid can have a first demand state (e.g., during off-peak hours) and a second demand state (e.g., during peak hours) greater than the first demand state. The power plant system can be controlled to have a first operating state in which (a) the steam from a first subset of the SMRs is routed to the electrical power conversion system to generate electricity that is routed to the power grid and (b) the steam from a second subset of the SMRs is routed to the sodium formate production system for use in producing the sodium formate and/or to the electrical power conversion system to generate electricity that is routed to the sodium formate production system for use in producing the sodium formate (e.g., by routing steam and electricity to the desalination plant and/or the sodium formate production plant). During the second demand state of the power grid, the power plant system can be controlled to have a second operating state different than the first operating state in which the steam from at least one of the SMRs in the second subset is routed to the electrical power conversion system to generate electricity that is routed to the power grid.

In some embodiments, the sodium formate can be shipped to a location remote from the power plant system and the sodium formate production system. The sodium formate can be processed to produce hydrogen as an energy carrier. The hydrogen can be utilized in a hydrogen fuel cell to regenerate electricity during, for example, the second demand state to support the greater power needs of the power grid.

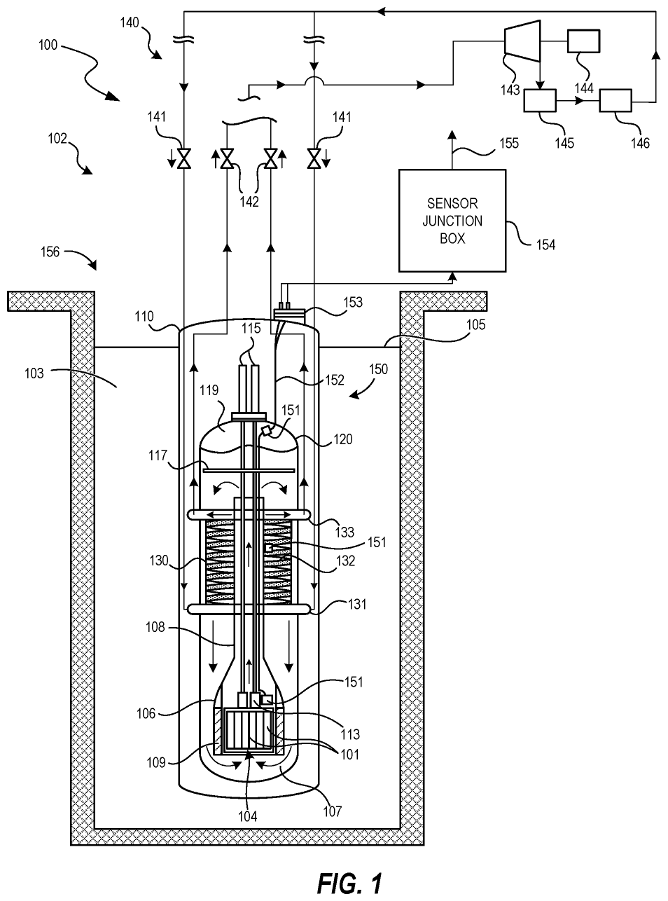

Certain details are set forth in the following description and in FIGS. 1-8C to provide a thorough understanding of various embodiments of the present technology. In other instances, well-known structures, materials, operations, and/or systems often associated with nuclear reactors, power plant systems, integrated energy systems, chemical production plants, industrial process plants, electrolysis systems, hydrogen and oxygen production plants, sodium formate production and processing, brine processing, and the like, are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth. The terminology used below is to be interpreted in its broadest reasonable manner, even though it is being used in conjunction with a detailed description of certain examples of embodiments of the technology.

The accompanying Figures depict embodiments of the present technology and are not intended to limit its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

To the extent any materials incorporated herein by reference conflict with the present disclosure, the present disclosure controls. The headings provided herein are for convenience only and should not be construed as limiting the subject matter disclosed.

### I. Select Embodiments of Nuclear Reactor Power Conversion Systems

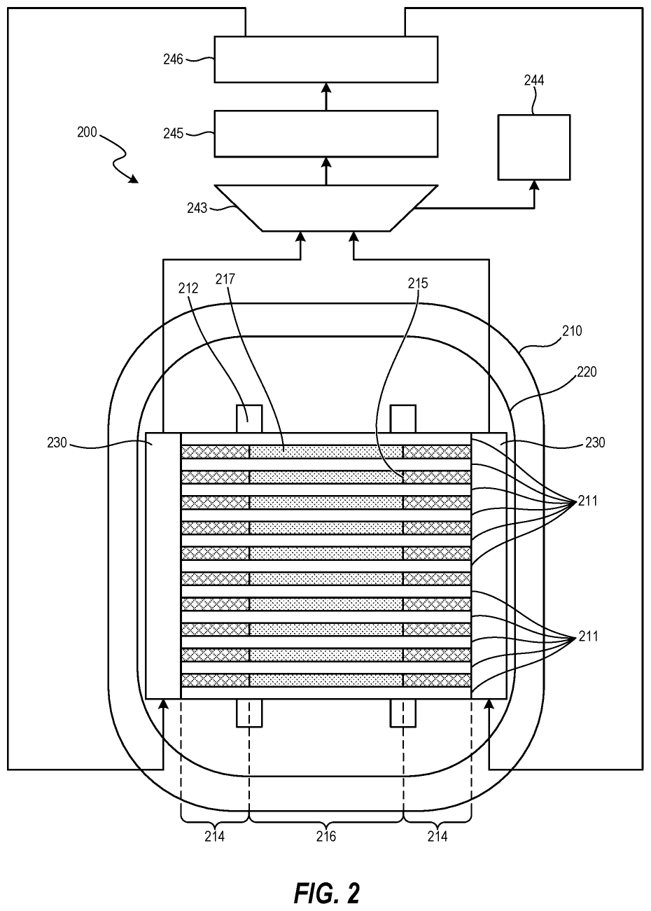

FIGS. 1 and 2 illustrate representative nuclear reactors that may be included in embodiments of the present technology. FIG. 1 is a partially schematic, partially cross-sectional view of a nuclear reactor system 100 configured in accordance with embodiments of the present technology. The system 100 can include a power module 102 having a reactor core 104 in which a controlled nuclear reaction takes place. Accordingly, the reactor core 104 can include one or more fuel assemblies 101. The fuel assemblies 101 can include fissile and/or other suitable materials. Heat from the reaction generates steam at a steam generator 130, which directs the steam to a power conversion system 140. The power conversion system 140 generates electrical power, and/or provides other useful outputs, such as super-heated steam. A sensor system 150 is used to monitor the operation of the power module 102 and/or other system components. The data obtained from the sensor system 150 can be used in real time to control the power module 102, and/or can be used to update the design of the power module 102 and/or other system components.

The power module 102 includes a containment vessel 110 (e.g., a radiation shield vessel, or a radiation shield container) that houses/encloses a reactor vessel 120 (e.g., a reactor pressure vessel, or a reactor pressure container), which in turn houses the reactor core 104. The containment vessel 110 can be housed in a power module bay 156. The power module bay 156 can contain a cooling pool 103 filled with water and/or another suitable cooling liquid. The bulk of the power module 102 can be positioned below a surface 105 of the cooling pool 103. Accordingly, the cooling pool 103 can operate as a thermal sink, for example, in the event of a system malfunction.

A volume between the reactor vessel 120 and the containment vessel 110 can be partially or completely evacuated to reduce heat transfer from the reactor vessel 120 to the surrounding environment (e.g., to the cooling pool 103). However, in other embodiments the volume between the reactor vessel 120 and the containment vessel 110 can be at least partially filled with a gas and/or a liquid that increases heat transfer between the reactor vessel 120 and the containment vessel 110. For example, the volume between the reactor vessel 120 and the containment vessel 110 can be at least partially filled (e.g., flooded with the primary coolant 107) during an emergency operation.

Within the reactor vessel 120, a primary coolant 107 conveys heat from the reactor core 104 to the steam generator 130. For example, as illustrated by arrows located within the reactor vessel 120, the primary coolant 107 is heated at the reactor core 104 toward the bottom of the reactor vessel 120. The heated primary coolant 107 (e.g., water with or without additives) rises from the reactor core 104 through a core shroud 106 and to a riser tube 108. The hot, buoyant primary coolant 107 continues to rise through the riser tube 108, then exits the riser tube 108 and passes downwardly through the steam generator 130. The steam generator 130 includes a multitude of conduits 132 that are arranged circumferentially around the riser tube 108, for example, in a helical pattern, as is shown schematically in FIG. 1. The descending primary coolant 107 transfers heat to a secondary coolant (e.g., water) within the conduits 132, and descends to the bottom of the reactor vessel 120 where the cycle begins again. The cycle can be driven by the changes in the buoyancy of the primary coolant 107, thus reducing or eliminating the need for pumps to move the primary coolant 107.

The steam generator 130 can include a feedwater header 131 at which the incoming secondary coolant enters the steam generator conduits 132. The secondary coolant rises through the conduits 132, converts to vapor (e.g., steam), and is collected at a steam header 133. The steam exits the steam header 133 and is directed to the power conversion system 140.

The power conversion system 140 can include one or more steam valves 142 that regulate the passage of high pressure, high temperature steam from the steam generator 130 to a steam turbine 143. The steam turbine 143 converts the thermal energy of the steam to electricity via a generator 144. The low-pressure steam exiting the turbine 143 is condensed at a condenser 145, and then directed (e.g., via a pump 146) to one or more feedwater valves 141. The feedwater valves 141 control the rate at which the feedwater re-enters the steam generator 130 via the feedwater header 131. In other embodiments, the steam from the steam generator 130 can be routed for direct use in an industrial process, such as a hydrogen and oxygen production plant, a chemical production plant, and/or the like, as described in detail below. Accordingly, steam exiting the steam generator 130 can bypass the power conversion system 140.

The power module 102 includes multiple control systems and associated sensors. For example, the power module 102 can include a hollow cylindrical reflector 109 that directs neutrons back into the reactor core 104 to further the nuclear reaction taking place therein. Control rods 113 are used to modulate the nuclear reaction, and are driven via fuel rod drivers 115. The pressure within the reactor vessel 120 can be controlled via a pressurizer plate 117 (which can also serve to direct the primary coolant 107 downwardly through the steam generator 130) by controlling the pressure in a pressurizing volume 119 positioned above the pressurizer plate 117.

The sensor system 150 can include one or more sensors 151 positioned at a variety of locations within the power module 102 and/or elsewhere, for example, to identify operating parameter values and/or changes in parameter values. The data collected by the sensor system 150 can then be used to control the operation of the system 100, and/or to generate design changes for the system 100. For sensors positioned within the containment vessel 110, a sensor link 152 directs data from the sensors to a flange 153 (at which the sensor link 152 exits the containment vessel 110) and directs data to a sensor junction box 154. From there, the sensor data can be routed to one or more controllers and/or other data systems via a data bus 155.

FIG. 2 is a partially schematic, partially cross-sectional view of a nuclear reactor system 200 (“system 200”) configured in accordance with additional embodiments of the present technology. In some embodiments, the system 200 can include some features that are at least generally similar in structure and function, or identical in structure and function, to the corresponding features of the system 100 described in detail above with reference to FIG. 1, and can operate in a generally similar or identical manner to the system 100.

In the illustrated embodiment, the system 200 includes a reactor vessel 220 and a containment vessel 210 surrounding/enclosing the reactor vessel 220. In some embodiments, the reactor vessel 220 and the containment vessel 210 can be roughly cylinder-shaped or capsule-shaped. The system 200 further includes a plurality of heat pipe layers 211 within the reactor vessel 220. In the illustrated embodiment, the heat pipe layers 211 are spaced apart from and stacked over one another. In some embodiments, the heat pipe layers 211 can be mounted/secured to a common frame 212, a portion of the reactor vessel 220 (e.g., a wall thereof), and/or other suitable structures within the reactor vessel 220. In other embodiments, the heat pipe layers 211 can be directly stacked on top of one another such that each of the heat pipe layers 211 supports and/or is supported by one or more of the other ones of the heat pipe layers 211.

In the illustrated embodiment, the system 200 further includes a shield or reflector region 214 at least partially surrounding a core region 216. The heat pipes layers 211 can be circular, rectilinear, polygonal, and/or can have other shapes, such that the core region 216 has a corresponding three-dimensional shape (e.g., cylindrical, spherical). In some embodiments, the core region 216 is separated from the reflector region 214 by a core barrier 215, such as a metal wall. The core region 216 can include one or more fuel sources, such as fissile material, for heating the heat pipes layers 211. The reflector region 214 can include one or more materials configured to contain/reflect products generated by burning the fuel in the core region 216 during operation of the system 200. For example, the reflector region 214 can include a liquid or solid material configured to reflect neutrons and/or other fission products radially inward toward the core region 216. In some embodiments, the reflector region 214 can entirely surround the core region 216. In other embodiments, the reflector region 214 may partially surround the core region 216. In some embodiments, the core region 216 can include a control material 217, such as a moderator and/or coolant. The control material 217 can at least partially surround the heat pipe layers 211 in the core region 216 and can transfer heat therebetween.

In the illustrated embodiment, the system 200 further includes at least one heat exchanger 230 (e.g., a steam generator) positioned around the heat pipe layers 211. The heat pipe layers 211 can extend from the core region 216 and at least partially into the reflector region 214, and are thermally coupled to the heat exchanger 230. In some embodiments, the heat exchanger 230 can be positioned outside of or partially within the reflector region 214. The heat pipe layers 211 provide a heat transfer path from the core region 216 to the heat exchanger 230. For example, the heat pipe layers 211 can each include an array of heat pipes that provide a heat transfer path from the core region 216 to the heat exchanger 230. When the system 200 operates, the fuel in the core region 216 can heat and vaporize a fluid within the heat pipes in the heat pipe layers 211, and the fluid can carry the heat to the heat exchanger 230. The heat pipes in the heat pipe layers 211 can then return the fluid toward the core region 216 via wicking, gravity, and/or other means to be heated and vaporized once again.

In some embodiments, the heat exchanger 230 can be similar to the steam generator 130 of FIG. 1 and, for example, can include one or more helically-coiled tubes that wrap around the heat pipe layers 211. The tubes of the heat exchanger 230 can include or carry a working fluid (e.g., a coolant such as water or another fluid) that carries the heat from the heat pipe layers 211 out of the reactor vessel 220 and the containment vessel 210 for use in generating electricity, steam, and/or the like. For example, in the illustrated embodiment the heat exchanger 230 is operably coupled to a turbine 243, a generator 244, a condenser 245, and a pump 246. As the working fluid within the heat exchanger 230 increases in temperature, the working fluid may begin to boil and vaporize. The vaporized working fluid (e.g., steam) may be used to drive the turbine 243 to convert the thermal potential energy of the working fluid into electrical energy via the generator 244. The condenser 245 can condense the working fluid after it passes through the turbine 243, and the pump 246 can direct the working fluid back to the heat exchanger 230 where it can begin another thermal cycle. In other embodiments, steam from the heat exchanger 230 can be routed for direct use in an industrial process, such as an enhanced oil recovery operation described in detail below. Accordingly, steam exiting the heat exchanger 230 can bypass the turbine 243, the generator 244, the condenser 245, the pump 246, etc.

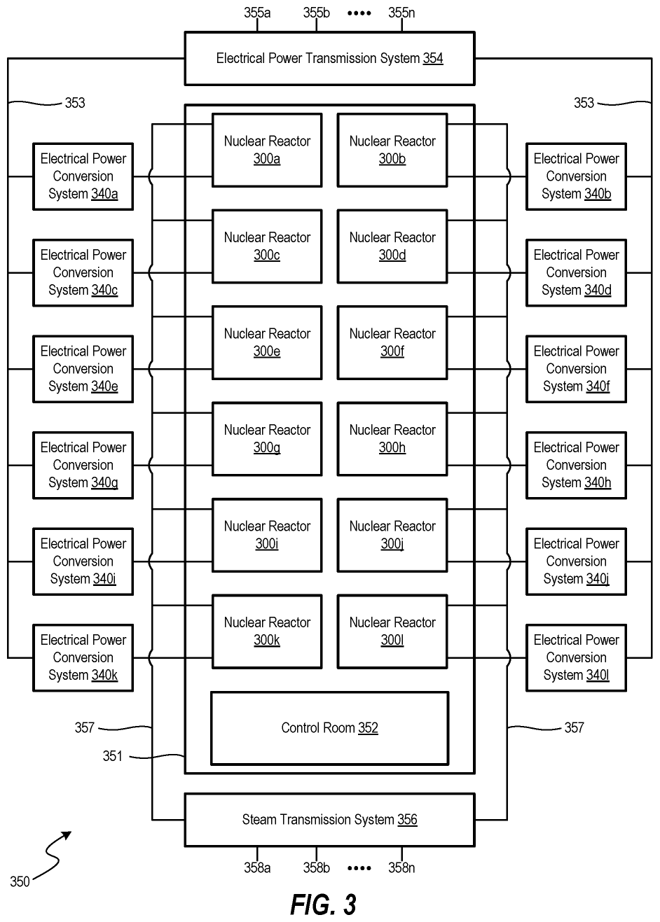

FIG. 3 is a schematic view of a nuclear power plant system 350 (“power plant system 350”) including multiple nuclear reactors 300 (individually identified as first through twelfth nuclear reactors 300a-1, respectively) in accordance with embodiments of the present technology. Each of the nuclear reactors 300 can be similar to or identical to the nuclear reactor 100 and/or the nuclear reactor 200 described in detail above with reference to FIGS. 1 and 2. The power plant system 350 can be “modular” in that each of the nuclear reactors 300 can be operated separately to provide an output, such as electricity or steam. The power plant system 350 can include fewer than twelve of the nuclear reactors 300 (e.g., two, three, four, five, six, seven, eight, nine, ten, or eleven of the nuclear reactors 300), or more than twelve of the nuclear reactors 300. The power plant system 350 can be a permanent installation or can be mobile (e.g., mounted on a truck, tractor, mobile platform, and/or the like). In the illustrated embodiment, each of the nuclear reactors 300 can be positioned within a common housing 351, such as a reactor plant building, and controlled and/or monitored via a control room 352.

Each of the nuclear reactors 300 can be coupled to a corresponding electrical power conversion system 340 (individually identified as first through twelfth electrical power conversion systems 340a-1, respectively). The electrical power conversion systems 340 can include one or more devices that generate electrical power or some other form of usable power from steam generated by the nuclear reactors 300. For example, the electrical power conversion systems 340 can include features that are similar or identical to the power conversion system 140 described in detail above with reference to FIG. 1. In some embodiments, multiple ones of the nuclear reactors 300 can be coupled to the same one of the electrical power conversion systems 340 and/or one or more of the nuclear reactors 300 can be coupled to multiple ones of the electrical power conversion systems 340 such that there is not a one-to-one correspondence between the nuclear reactors 300 and the electrical power conversion systems 340.

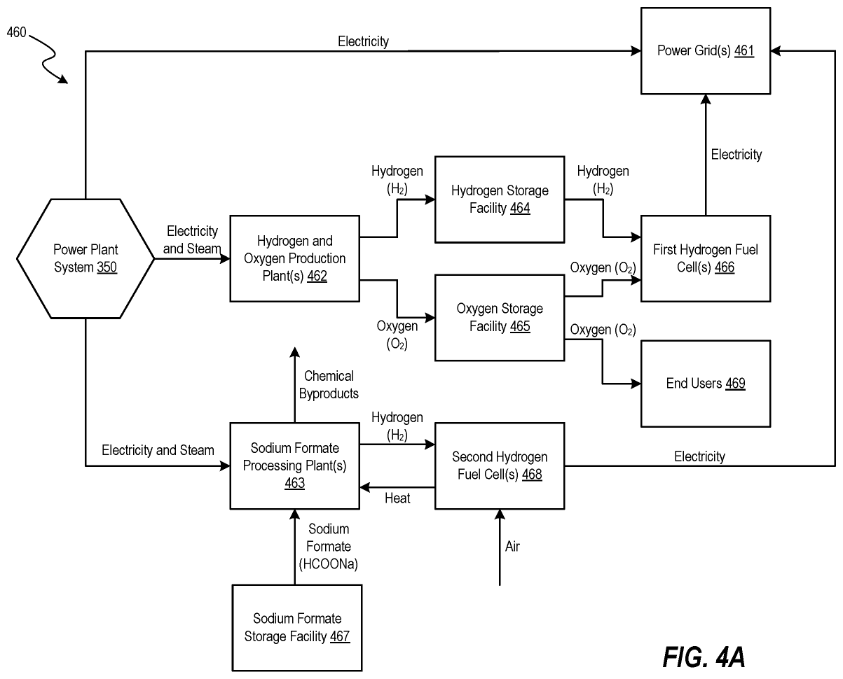

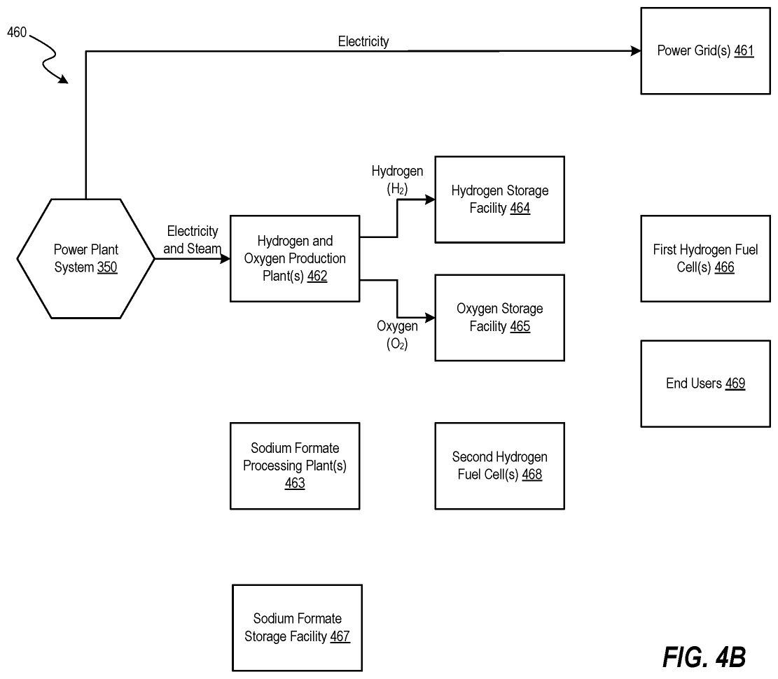

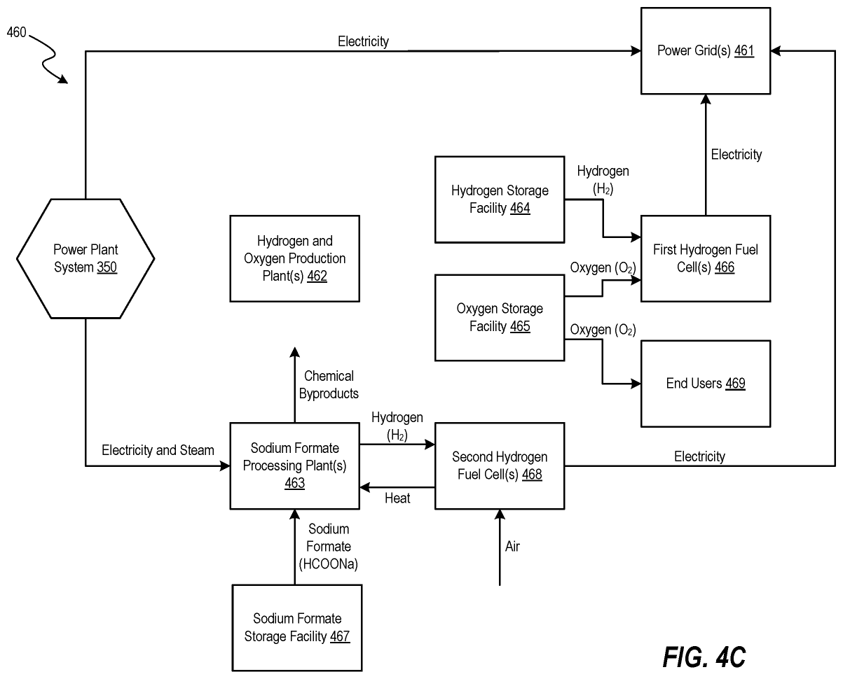

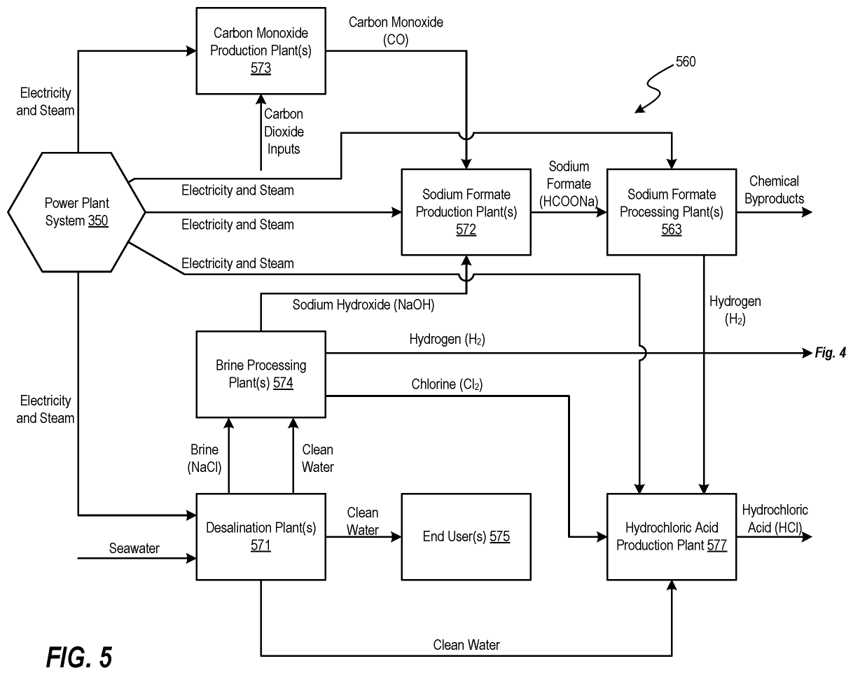

The electrical power conversion systems 340 can be further coupled to an electrical power transmission system 354 via, for example, an electrical power bus 353. The electrical power transmission system 354 and/or the electrical power bus 353 can include one or more transmission lines, transformers, and/or the like for regulating the current, voltage, and/or other characteristic(s) of the electricity generated by the electrical power conversion systems 340. The electrical power transmission system 354 can route electricity via a plurality of electrical output paths 355 (individually identified as electrical output paths 355a-n) to one or more end users and/or end uses, such as different electrical loads of an integrated energy system as described in greater detail below with reference to FIGS. 4A-8C.

Each of the nuclear reactors 300 can further be coupled to a steam transmission system 356 via, for example, a steam bus 357. The steam bus 357 can route steam generated from the nuclear reactors 300 to the steam transmission system 356 which in turn can route the steam via a plurality of steam output paths 358 (individually identified as steam output paths 358a-n) to one or more end users and/or end uses, such as different steam inputs of an integrated energy system as described in greater detail below with reference to FIGS. 4A-8C.

In some embodiments, the nuclear reactors 300 can be individually controlled (e.g., via the control room 352) to provide steam to the steam transmission system 356 and/or steam to the corresponding one of the electrical power conversion systems 340 to provide electricity to the electrical power transmission system 354. In some embodiments, the nuclear reactors 300 are configured to provide steam either to the steam bus 357 or to the corresponding one of the electrical power conversion systems 340, and can be rapidly and efficiently switched between providing steam to either. Accordingly, in some aspects of the present technology the nuclear reactors 300 can be modularly and flexibly controlled such that the power plant system 350 can provide differing levels/amounts of electricity via the electrical power transmission system 354 and/or steam via the steam transmission system 356. For example, where the power plant system 350 is used to provide electricity and steam to one or more industrial process—such as various components of the integrated energy systems described in the detail below with reference to FIGS. 4A-8C—the nuclear reactors 300 can be controlled to meet the differing electricity and steam requirements of the industrial processes.

As one example, during a first operational state of an integrated energy system employing the power plant system 350, a first subset of the nuclear reactors 300 (e.g., the first through sixth nuclear reactors 300a-f) can be configured to provide steam to the steam transmission system 356 for use in the first operational state of the integrated energy system, while a second subset of the nuclear reactors 300 (e.g., the seventh through twelfth nuclear reactors 300g-l) can be configured to provide steam to the corresponding ones of the electrical power conversion systems 340 (e.g., the seventh through twelfth electrical power conversion systems 340g-l) to generate electricity for the first operational state of the integrated energy system. Then, during a second operational state of the integrated energy system when a different (e.g., greater or lesser) amount of steam and/or electricity is required, some or all the first subset of the nuclear reactors 300 can be switched to provide steam to the corresponding ones of the electrical power conversion systems 340 (e.g., the seventh through twelfth electrical power conversion systems 340g-l) and/or some or all of the second subset of the nuclear reactors 300 can be switched to provide steam to the steam transmission system 356 to vary the amount of steam and electricity produced to match the requirements/demands of the second operational state. Other variations of steam and electricity generation are possible based on the needs of the integrated energy system. That is, the nuclear reactors 300 can be dynamically/flexibly controlled during other operational states of an integrated energy system to meet the steam and electricity requirements of the operational state.

In contrast, some conventional nuclear power plant systems can typically generate either steam or electricity for output, and cannot be modularly controlled to provide varying levels of steam and electricity for output. Moreover, it is typically difficult (e.g., expensive, time consuming, etc.) to switch between steam generation and electricity generation in conventional nuclear power plant systems. Specifically, for example, it is typically extremely time consuming to switch between steam generation and electricity generation in prototypical large nuclear power plant systems.

The nuclear reactors 300 can be individually controlled via one or more operators and/or via a computer system. Accordingly, many embodiments of the technology described herein may take the form of computer- or machine- or controller-executable instructions, including routines executed by a programmable computer or controller. Those skilled in the relevant art will appreciate that the technology can be practiced on computer/controller systems other than those shown and described herein. The technology can be embodied in a special-purpose computer, controller or data processor that is specifically programmed, configured or constructed to perform one or more of the computer-executable instructions described below. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, mini computers and the like). Information handled by these computers can be presented at any suitable display medium, including a liquid crystal display (LCD).

The technology can also be practiced in distributed environments, where tasks or modules are performed by remote processing devices that are linked through a communications network. In a distributed computing environment, program modules or subroutines may be located in local and remote memory storage devices. Aspects of the technology described herein may be stored or distributed on computer-readable media, including magnetic or optically readable or removable computer disks, as well as distributed electronically over networks. Data structures and transmissions of data particular to aspects of the technology are also encompassed within the scope of the embodiments of the technology.

### II. Select Embodiments of Integrated Energy Systems

The power plant system 350 of FIG. 3 can be coupled to one or more industrial processes and/or systems to form an integrated energy system for producing green (e.g., carbon-free or reduced-carbon) hydrogen, sodium formate, and/or other industrial products. For example, electricity and/or steam can be diverted from the power plant system 350 to produce hydrogen as an energy carrier for short-term storage during off-peak hours to support the energy imbalance market (EIM). Typically, liquid organic hydrogen carriers (LOHCs) can be used to support this period with a predictable rate of hydrogenation and dehydrogenation. The release of the stored hydrogen can be fed into a reversible solid oxide electrolysis cell (RSOEC) and/or other hydrogen fuel cell for the generation of electricity. However, such an EIM segment can be volatile and unpredictable such that the amount of hydrogen available for electricity production is insufficient. Therefore, an in-situ, on-demand, hydrogen production mechanism can be beneficial to support the EIM and also for emergency needs. During the EIM, thermal-chemical process can be utilized to release hydrogen from sodium formate when there is a demand for electricity that is larger than the energy from the stored hydrogen. Some embodiments of the present technology can use the power plant system 350—in which both electricity and thermal energy are abundantly available—to process sodium formate to generate hydrogen.

FIG. 4A is a schematic diagram of an integrated energy system 460 including the power plant system 350 of FIG. 3 in accordance with embodiments of the present technology. In the illustrated embodiment, the power plant system 350 is configured for use in an industrial process/operation and, more particularly, for use in producing hydrogen as an energy carrier and subsequently consuming the hydrogen to generate electricity to, for example, support the energy imbalance market (EIM). Specially, the power plant system 350 is configured to generate electricity and route the electricity (e.g., via one or more power lines, via the electrical power transmission system 354 of FIG. 3) to one or more power grids 461, one or more hydrogen and oxygen production plants 462, and one or more sodium formate processing plants 463. The power plant system 350 is further configured to generate steam and route the steam (e.g., via one or more steam transmission lines, via the steam transmission system 356 of FIG. 3) to the hydrogen and oxygen production plant 462 and the sodium formate processing plant 463.

The power grid 461 can supply power to a plurality of remote end users, or can be dedicated to a specific consumer. The power plant system 350 can be a permanent or temporary installation built at or near the location of the hydrogen and oxygen production plant 462 and/or the sodium formate processing plant 463, or can be a mobile or partially mobile system that is moved to and assembled at or near the location of the hydrogen and oxygen production plant 462 and the sodium formate processing plant 463. More generally, the power plant system 350 can be local (e.g., positioned at or near) the industrial processes/operations it supports, such as the hydrogen and oxygen production plant 462 and the sodium formate processing plant 463. For example, the power plant system 350 can be located within 0.4 km (0.25 mile), within 0.8 km (0.5 mile), within 3.22 km (2 miles), within 4.82 km (3 miles), or within 8.1 km (5 miles) of the industrial processes/operations it supports. In some embodiments, the power plant system 350 includes four, six, twelve, or a different number of the nuclear reactors 300 (FIG. 3) and has a power output of between 308-924 megawatts electrical (MWe). In some embodiments, the power plant system 350 can output between about 308-462 MWe.

The hydrogen and oxygen production plant 462 can utilize one or more electrolysis processes to generate hydrogen and oxygen, such as a high temperature steam electrolysis (HTSE) process and/or with liquid alkaline (LA; e.g., alkaline water) electrolysis or proton exchange membrane (PEM) water electrolysis (low-temperature electrolysis (“LTE”)). In some embodiments, the hydrogen and oxygen production plant 462 utilizes an HTSE process, and the integrated energy system 460 can further include an auxiliary heater (not shown) that receives steam and electricity from the power plant system 350 and utilizes the electricity from the power plant system 350 to superheat the steam from the power plant system 350 (e.g., to between 300-850° C., to between 700-850° C., to above 600° C., to 850° C., to above 850° C.) for use in the HTSE process. In some embodiments, the hydrogen and oxygen production plant 462 can utilize a LTE process, and the integrated energy system 460 can further include a water production plant (not shown) that receives electricity from the power plant system 350 and utilizes the electricity to generate high-quality water (e.g., by demineralizing and/or otherwise removing contaminants and/or unwanted material from a water source) for use in the LTE process. Further details of hydrogen and oxygen production plants that utilize HTSE and/or LTE processes to generate hydrogen and oxygen are described in U.S. patent application Ser. No. 18/116,819, titled “SMALL MODULAR NUCLEAR REACTOR INTEGRATED ENERGY SYSTEMS FOR ENERGY PRODUCTION AND GREEN INDUSTRIAL APPLICATIONS,” and filed Mar. 2, 2023, which is incorporated by reference herein in its entirety.

In the illustrated embodiment, the integrated energy system 460 further comprises one or more hydrogen storage facilities 464 and one or more oxygen storage facilities 465 configured to receive and store hydrogen (H₂) and oxygen (O₂), respectively, generated by the hydrogen and oxygen production plant 462. The hydrogen storage facility 464 can store the hydrogen as a gas and/or can store the hydrogen with one or more liquid organic hydrogen carriers (LOHCs) that can absorb and release the hydrogen through chemical reactions, and/or via another storage media. The LOHCs can have a predictable rate of hydrogenation and dehydrogenation. Oxygen stored at the oxygen storage facility 465 can be routed to (e.g., shipped, transported) and utilized by one or more end users 469. The end users 469 can comprise industrial processing plants, hospitals, and/or the like.

In some embodiments, the integrated energy system 460 further includes one or more first hydrogen fuel cells 466 positioned to receive hydrogen and oxygen from the hydrogen storage facility 464 and the oxygen storage facility 465, respectively. The first hydrogen fuel cell 466 can convert the hydrogen to electricity for routing to the power grid 461 and/or to another component of the integrated energy system 460. The first hydrogen fuel cell 466 can comprise a reversible solid oxide fuel cell (RSOFC) and/or the like that uses an electrochemical process to convert hydrogen into electricity.

The sodium formate processing plant 463 is configured to receive sodium formate from a sodium formate storage facility 467 and/or other source and utilize the electricity and/or steam from the power plant system 350 to process the sodium formate to produce hydrogen and other chemical byproducts. For example, the sodium formate processing plant 463 can comprise a sodium formate reaction chamber in which the sodium formate and subsequent reaction products can be heated and allowed to react to produce hydrogen. Upon heating, sodium formate (HCOONa) decomposes to form disodium oxalate ((COO)₂Na₂) and hydrogen gas (H₂) according to equation (1) below:

2HCOONa→(COO)₂Na₂+H₂ (1)

Hydrogen is released when the temperature is kept below 290° C. When the temperature is increased to above 290° C., sodium carbonate (Na₂CO₃) will form and carbon monoxide (CO) is released according to equation (2) below:

(COO)₂Na₂→Na₂CO₃+CO (2)

Therefore, in some aspects of the present technology the temperature can be maintained below 300° C. to minimize the production of carbon monoxide to support the EIM scenarios.

If additional quantity of hydrogen is required to support additional power demand, the sodium formate processing plant 463 can receive super-heated steam (>750° C.) from the power plant system 350 and/or heated by heat generated by the second hydrogen fuel cell 468, and utilize the super-heated system (e.g., by injecting the steam into the sodium formate reaction chamber) in a catalytic steam reforming process, equation (2) from above, to react with the carbon monoxide such that additional hydrogen (H₂) is released and carbon dioxide (CO₂) is formed according to equation (3) below:

CO+H₂O→CO₂+H₂ (more hydrogen) (3)

The sodium formate processing plant 463 can further allow the carbon dioxide from the catalytic steam reforming process to react with the sodium carbonate and additional steam from the power plant system 350 to produce sodium bicarbonate (NaHCO₃; baking soda) according to equation (4) below combining equations (2) and (3) above:

Na₂CO₃+2CO₂+H₂O→2NaHCO₃ (4)

Accordingly, the sodium formate processing plant 463 can utilize the electricity and steam from the power plant system 350 to process the sodium formate to produce hydrogen and other chemical byproducts such as sodium carbonate (according to equation (2) above) and/or sodium bicarbonate (according to equation (4) above). In some aspects of the present technology, the carbon dioxide produced in equation (3) is not released and becomes part of the finally produced sodium bicarbonate in equation (4)—reducing or eliminating entirely carbon dioxide emissions. The sodium carbonate, sodium bicarbonate, and/or other chemical byproducts (e.g., sodium oxalate) can be utilized for other industrial processes. In some embodiments, from the above equations (1)-(4), 136 grams of sodium formate can be utilized to produce 4 grams of hydrogen based on molar equations.

In some embodiments, the integrated energy system 460 further includes one or more second (e.g., reserve) hydrogen fuel cells 468 positioned to receive the hydrogen produced by the sodium formate processing plant 463 and air (and/or oxygen from the oxygen storage facility 465) and configured to generate electricity therefrom. The second hydrogen fuel cell 468 can convert the hydrogen to electricity for routing to the power grid 461 and/or another component of the integrated energy system 460. The second hydrogen fuel cell 468 can be generally similar or identical to the first hydrogen fuel cell and uses an electrochemical process to convert hydrogen into electricity. In some embodiments, the first hydrogen fuel cell 466 and the second hydrogen fuel cell 468 can comprise the same fuel cell and/or can be integrated into a common system, location, etc.

In some embodiments, the second hydrogen fuel cell 468 generates process heat that can be utilized by the sodium formate processing plant 463 for use in processing the sodium formate to produce hydrogen (e.g., in any of the reactions given above by equations (1)-(4)) in addition to or alternatively to heat and/or steam from the power plant system 350. For example, heat generated from the electrical and/or steam outputs of the power plant system 350 can be used upon initial startup of the sodium formate processing plant 463 and then heat generated from the second hydrogen fuel cell 468 can be used to sustain the sodium formate reaction during further operation. Accordingly, in some aspects of the present technology the sodium formate processing plant 463 only requires input steam and/or electricity from the power plant system 350 to achieve startup, and is sustained by the second hydrogen fuel cell 468 thereafter.

Sodium formate is a salt that can be transported and stored (e.g., at the sodium formate storage facility 467) easily and safely. Moreover, sodium formate can be produced at large-scale inexpensively from formic acid via carbonylation of methanol followed by adding water to the methyl formate, by neutralizing formic acid with sodium hydroxide, and/or by other processes, such as those described in detail below with reference to FIG. 5. Additionally, the sodium formate can be processed at relatively low temperatures (e.g., less than 290° C., about 250° C.) according to equation (1) above to produce hydrogen in-situ and on-demand.

In some embodiments, the power plant system 350 can be controlled to selectively provide differing amounts of electricity and/or steam to (i) the power grid 461, (ii) the hydrogen and oxygen production plant 462, and (iii) the sodium formate processing plant 463 based on the demands of the power grid 461 to support an energy imbalance market (EIM). For example, FIGS. 4B and 4C are schematic diagrams of the integrated energy system 460 configured during off-peak hours (e.g., a first demand state of the power grid 461) and during peak hours (e.g., typically during the 4-hour period between 6 pm to 10 pm; e.g., a second demand state of the power grid 461 greater than the first demand state of the power grid 461), respectively, in accordance with embodiments of the present technology.

Referring to FIG. 4B, during off-peak hours the power plant system 350 can be controlled/configured to have a first operating state in which the power plant system 350 provides excess electricity and steam to the hydrogen and oxygen production plant 462 to generate hydrogen (as an energy carrier) and oxygen for short term storage at the hydrogen storage facility 464 and the oxygen storage facility 465, respectively. The sodium formate processing plant 463 may not process sodium formate during the off-peak hours, and the first hydrogen fuel cell 466 and the second hydrogen fuel cell 468 may be offline (e.g., not producing electricity).

Referring to FIG. 4C, during peak-hours (e.g., typically during the 4-hour period between 6 pm to 10 pm), the power plant system 350 can be controlled/configured to have a second operating state different than the first operating state in which more of the electricity is routed from the power plant system 350 to the power grid 461 while the first hydrogen fuel cell 466 is operated to produce additional electricity from the hydrogen produced during the off-peak hours and stored at the hydrogen storage facility 464. Likewise, some electricity and/or steam can be routed to the sodium formate processing plant 463 to process sodium formate to produce hydrogen for use in generating electricity via the second hydrogen fuel cell 468. As described above, the electricity and/or steam from the power plant system 350 can be used to initialize the sodium formate reaction before heat from the second hydrogen fuel cell 468 subsequently supplies some or all of the heat needed to process the sodium formate.

Accordingly, referring to FIGS. 4A-4C, the integrated energy system 460 can (i) route excess steam and electricity from the power plant system 350 to the hydrogen and oxygen production plant 462 to produce hydrogen during off-peak hours when the demand of the power grid 461 is less and then (ii) utilize the produced hydrogen to regenerate electricity via the first hydrogen fuel cell 466 to meet the greater demand of the power grid 461 during peak hours and/or other unexpected sudden requirements. At the same time, the power plant system 350 can be controlled to deliver electricity and/or steam to the sodium formate processing plant 463 during peak hours to generate hydrogen in-situ and on-demand that can be fed to the second hydrogen fuel cell 468 to generate electricity for the power grid 461 to support inadequate energy production during peak hours and/or other unexpected sudden requirements.

In some aspects of the present technology, some of nuclear reactors 300 (FIG. 3) of the power plant system 350 can be dynamically switched from producing electricity for routing to the power grid 461 to producing steam for use in the hydrogen and oxygen production plant 462 and/or the sodium formate processing plant 463. Accordingly, the modularity of the nuclear reactors 300 allows the power plant system 350 to flexibly/dynamically switch the output of electricity and steam from individual ones of the nuclear reactors 300 based on the demands of the integrated energy system 460 (e.g., the power grid 461).

Additionally, one or more of the nuclear reactors 300 can be individually taken offline for servicing, maintenance, refueling, etc., while the remainder of the nuclear reactors 300 can continue to produce steam and/or electricity. Accordingly, the power plant system 350 can continue to provide steam and/or electricity to the power grid 461, the hydrogen and oxygen production plant 462, and/or the sodium formate processing plant 463, even during servicing, maintenance, refueling, etc. In contrast, conventional nuclear reactor systems must be entirely shut down during such procedures such that neither steam nor electricity are available.

In some aspects of the present technology, the integrated energy system 460 can be highly efficient and produce little or no carbon emissions. In contrast, conventional systems for producing hydrogen and oxygen generally rely on steam-methane reforming which reacts natural gas with steam at elevated temperature to produce carbon monoxide and hydrogen. Steam-methane reforming has significant carbon emissions—generally producing about 9.3 kilograms (kg) of carbon dioxide per kg of hydrogen produced.

In some embodiments, an integrated energy system in accordance with the present technology can be configured to produce sodium formate in addition to or alternatively to processing the sodium formate to generate hydrogen as an energy carrier. FIG. 5, for example, is a schematic diagram of an integrated energy system 560 including the power plant system 350 of FIG. 3 in accordance with additional embodiments of the present technology. In the illustrated embodiment, the integrated energy system 560 is generally configured to treat seawater or brackish water to produce brine and clean water, treat the brine to generate sodium hydroxide (NaOH) and hydrogen, and utilize the sodium hydroxide to generate sodium formate (HCOONa). The integrated energy system 560 can include some features that are at least generally similar in structure and function, or identical in structure and function, to the corresponding features of the integrated energy system 460 described in detail above with reference to FIGS. 4A-4C, and can operate in a generally similar or identical manner to the integrated energy system 460.

Specially, the power plant system 350 is configured to generate electricity and steam and route the electricity and steam (e.g., via one or more power lines, via the electrical power transmission system 354 of FIG. 3, via one or more steam transmission lines, via the steam transmission system 356 of FIG. 3) to one or more desalination plants 571, one or more sodium formate production plants 572, and one or more carbon monoxide production plants 573. In some embodiments, the power plant system 350 is further configured to route electricity to a power grid (not shown).

The power plant system 350 can be a permanent or temporary installation built at or near the location of the desalination plant 571, the sodium formate production plant 572, and/or the carbon monoxide production plant 573, or can be a mobile or partially mobile system that is moved to and assembled at or near the location of the desalination plant 571, the sodium formate production plant 572, and/or the carbon monoxide production plant 573. In some embodiments, the power plant system 350 can be a floating nuclear power plant and/or can be positioned at a coastal location near the desalination plant 571.

The desalination plant 571 can receive and process seawater or brackish water or other saline water to produce clean water (e.g., drinking water) and sodium chloride (NaCL), which is commonly referred to as brine, as a by-product. Accordingly, in some embodiments the desalination plant 571 can be positioned near a source of seawater or brackish water, such along the coastline of an arid country that lacks useful clean water for metropolitans and industries. The desalination plant 571 can treat the seawater or brackish water to produce clean water and brine using a distillation process (e.g., vacuum distillation, multi-stage flash distillation, multiple-effect distillation (MED), vapor-compression distillation, and/or the like), an osmosis process (e.g., reverse osmosis, forward osmosis), an electrodialysis process, and/or the like. For example, the desalination plant 571 can utilize a reverse osmosis process that uses semipermeable membranes and applied pressure (on the membrane feed side) to preferentially induce water permeation through the membrane while rejecting salts.

In the illustrated embodiment, some of the clean water is routed to one or more end users 575, such as homes, hospitals, industries, cities, etc. Brine is a high-concentration water solution of sodium chloride ranging from about 4% up to about 26%. Brine is denser than seawater or brackish water, and therefore will sink to the bottom of the ocean if released therein, which can damage the oceanic ecosystem. Studies have shown that dilution is not a solution because brine dispersal from desalination plants can travel several kilometers away, meaning that it has the potential to cause harm to ecosystems far away from the desalination plants. Brine in small quantity can be used for food processing and for de-icing of roads. But, due to the large volume that is generated from desalination processes, an innovative process to reprocess the brine is desired to ensure that a desalination process to produce clean water for consumption and for industrial usage can be encouraged and deployed.

In the illustrated embodiment, the integrated energy system 560 further comprises one or more brine processing plants 574 operably coupled to the desalination plant 571. The brine processing plant 574 is configured to receive brine and clean water from the desalination plant 571 and to process the brine to generate chlorine (Cl₂), hydrogen (H₂), and sodium hydroxide (NaOH). In some embodiments, the brine processing plant 574 is configured to treat the brine using an electrolysis process, such as the chlor-alkali membrane process, to produce sodium hydroxide from a solution of sodium chloride. The chlor-alkali membrane process can employ a membrane cell, and can be referred to as a membrane cell process. The membrane cell process can use a membrane cell to partition the brine solution so that Cl⁻ ions are inhibited or even prevented from migrating to the cathode side of the cell to react with the produced sodium hydroxide solution. The process can also simultaneously produce chlorine gas and hydrogen gas. The sodium hydroxide builds up at the cathode, where water is reduced to hydrogen gas and hydroxide ions (OH⁻) according to equation (5) below:

2Na⁺+2H₂O+2e⁻→H₂+2NaOH (5)

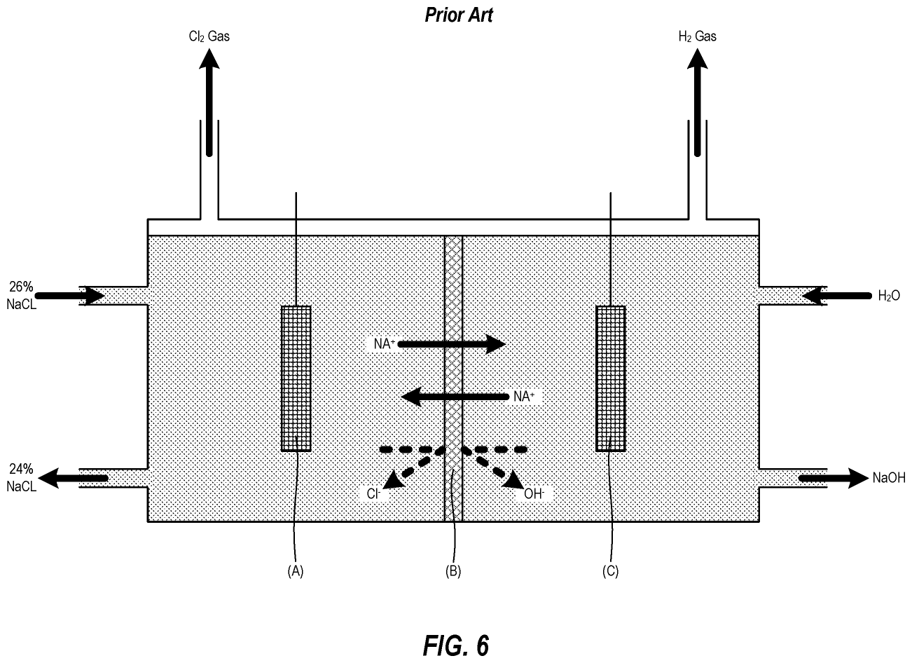

As a result, the sodium hydroxide typically can be collected at the cathode. More specifically, FIG. 6 is a schematic diagram of a membrane cell for carrying out the chlor-alkali membrane process via the electrolysis of aqueous sodium chloride (brine) in accordance with the prior art. The brine can be provided from the desalination plant 571 (FIG. 5). As shown, at an anode (A), chloride (Cl⁻) is oxidized to chlorine and chlorine gas is formed. An ion-selective membrane (B) allows the counter ion Nat to freely flow across, but inhibits or even prevents anions such as hydroxide (OH⁻) and chloride from diffusing across into the opposite sides. At a cathode (C), water (H₂O) is reduced to hydroxide and hydrogen gas. The water can be provided from the desalination plant 571 (FIG. 5).

The chemical reactions that take place are as follows. First, saturated brine is passed into a first chamber of the membrane cell where the chloride ions are oxidized at the anode (A), losing electrons to become chlorine gas according to equation (6) below:

2Cl⁻→Cl₂+2e⁻ (6)

Sodium ions (Na⁺) pass to the second chamber where they react with the hydroxide ions to produce caustic soda (NaOH). Positive hydrogen ions pulled from water molecules are reduced by the electrons provided by the electrolytic current, to hydrogen gas, releasing hydroxide ions into the solution according to equation (7) below:

2H₂O+2e⁻→H₂+2OH (7)

Accordingly, the prescribed overall reaction for the electrolysis of brine is given by equation (8) below:

2NaCl+2H₂O→Cl₂+H₂+2NaOH (8)

Referring again to FIG. 5, the carbon monoxide production plant 573 can receive electricity and steam from the power plant system 350 and carbon dioxide (CO₂) inputs and utilize the electricity and steam to generate carbon monoxide (MO). In some embodiments, the carbon monoxide production plant 573 can first capture and/or produce carbon dioxide (CO₂) before processing the carbon dioxide to produce carbon monoxide as an output. In some embodiments, the carbon dioxide inputs comprise atmospheric air and the carbon monoxide production plant 573 is configured to capture carbon dioxide from the air via a direct air capture (DAC) process. In other embodiments, the carbon dioxide inputs comprise bulk plastics and the carbon monoxide production plant 573 can gasify the bulk plastics to generate carbon dioxide. In some embodiments, the carbon monoxide production plant 573 processes the produced carbon dioxide via an electrolysis process to produce carbon monoxide. For example, the carbon monoxide production plant 573 can comprise a solid oxide electrolyzer cell (SOEC) and/or other solid oxide fuel cell that electrolyzes the carbon dioxide via a solid oxide or ceramic electrolyte to produce carbon monoxide. More specifically, CO 2 can be fed into a SOEC fuel stack cathode side with an applied current. On the anode side, oxygen or simply air, is fed into the SOEC stack to enhance the electrolysis process. The output stream from the cathode side of the SOEC stack contains a mixture of CO and CO₂ according to equation (9) below:

2CO₂→2CO+O₂ (9)

In some embodiments, the carbon monoxide production plant 573 can have some features generally similar to and/or can operate in a generally similar method to any of the methods/devices/systems for generating carbon monoxide from carbon dioxide described in (i) Hauch, A., et al. “Recent Advances in Solid Oxide Cell Technology for Electrolysis,” Science, vol. 370, no. 6513, 9 Oct. 2020, https://doi.org/10.1126/science.aba6118 and/or (ii) International Patent Application Publication No. WO2014/154253, titled “A PROCESS FOR PRODUCING CO FROM CO2 IN A SOLID OXIDE ELECTROLYSIS CELL,” and filed Mar. 26, 2013, each of which is incorporated herein by reference in its entirety.

The sodium formate production plant 572 is configured to receive (i) electricity and steam from the power plant system 350, (ii) carbon monoxide from the carbon monoxide production plant 573, and (iii) sodium hydroxide from the brine processing plant 574 and to utilize these components to produce sodium formate (HCOONa). In some embodiments, the sodium formate production plant 572 can utilize the electricity and/or steam from the power plant system to heat the carbon monoxide and sodium formate to about 130° C. and to pressurize the carbon monoxide and sodium formate to between about 6-8 bar (e.g., within a reaction chamber) such that the carbon monoxide is absorbed into the sodium hydroxide to produce solid sodium formate. The reaction is given by equation (10) below:

CO+NaOH→HCOONa (10)

In some embodiments, the sodium formate can be used to generate hydrogen as an energy carrier for use in, for example, regenerating electricity via a hydrogen fuel cell as described in detail above with reference to FIGS. 4A-4C. In the illustrated embodiment, for example, the integrated energy system 560 further comprises one or more sodium formate processing plants 563 which can utilize electricity and steam from the power plant system 350 (and/or another source) to process the sodium formate to produce hydrogen and other chemical byproducts such as sodium carbonate (according to equation (2) above), sodium bicarbonate (according to equation (4) above), sodium oxalate, and/or other chemicals. The resultant hydrogen produced by the sodium formate processing plant 563 and/or the hydrogen produced by the brine processing plant 574 can be used as an energy carrier to generate electricity as described in detail above with reference to FIGS. 4A-4C and/or for other purposes.

In some embodiments, the integrated energy system 560 further comprises one or more hydrochloric acid production plants 577. The hydrochloric acid production plant 577 can (i) receive hydrogen from the sodium formate processing plant 563 and/or hydrogen from the brine processing plant 574, chlorine from the brine processing plant 574, and clean water from the desalination plant 571 and (ii) utilize steam and/or electricity from the power plant system 350 (and/or another source) to process the hydrogen, chlorine, and water to generate hydrochloric acid (HCl).

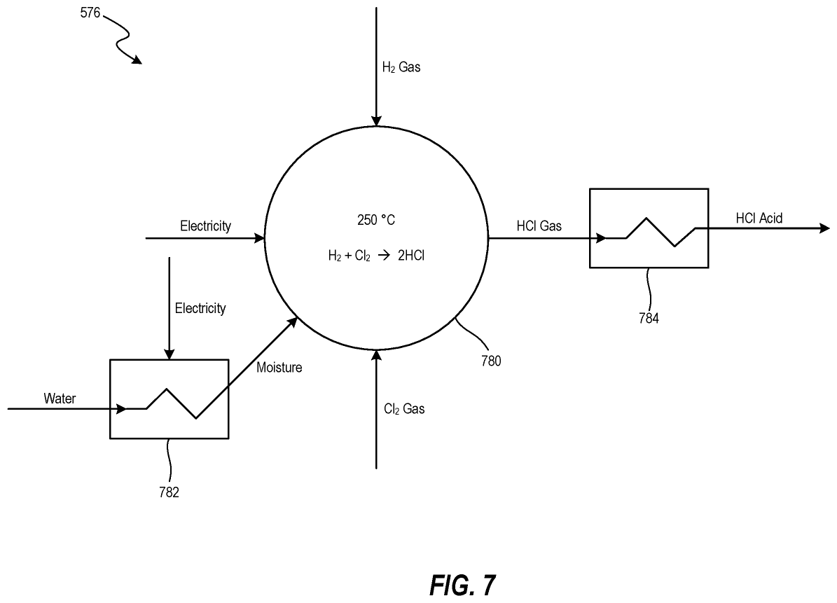

More specifically, FIG. 7 is a schematic diagram of a process carried out by the hydrochloric acid production plant 577 for producing hydrochloric acid in accordance with embodiments of the present technology. In the illustrated embodiment, the hydrochloric acid production plant 577 comprises a reaction chamber 780, an auxiliary heater 782, and a thermal energy recovery system 784. Referring to FIGS. 5 and 7, the reaction chamber 780 receives hydrogen gas (H₂) from the brine processing plant 574 and/or the sodium formate processing plant 563 and chlorine gas (Cl₂) from the brine processing plant 574. The reaction chamber 780 can further utilize electricity from the power plant system 350 to heat the reaction chamber to between about 230-270° C. (e.g., to about 250° C.) to react the hydrogen gas and chlorine gas to generate gaseous hydrogen chloride (HCl). In some embodiments, the auxiliary heater 782 can receive water from the desalination plant 571 and/or another source and heat the water utilizing electricity from the power plant system 350 to generate moisture (e.g., water vapor, steam) that is routed into the reaction chamber 780. In some aspects of the present technology, the moisture makes the reaction of the hydrogen gas and chlorine gas extremely efficient. The reaction of the hydrogen gas and chlorine gas is exothermic. Therefore, in some embodiments the thermal energy recovery system 784 can receive the gaseous hydrogen chloride and recover thermal energy therefrom while cooling the gaseous hydrogen chloride into hydrochloric acid. The hydrochloric acid can be stored and/or used in one or more industrial processes.

In some aspects of the present technology, some of nuclear reactors 300 (FIG. 3) of the power plant system 350 can be dynamically switched from producing electricity and/or steam for use in the desalination plant 571, the sodium formate production plant 572, the carbon monoxide production plant 573, the sodium formate processing plant 563, and/or the hydrochloric acid production plant 577. Accordingly, the modularity of the nuclear reactors 300 allows the power plant system 350 to flexibly/dynamically switch the output of electricity and steam from individual ones of the nuclear reactors 300 based on the demands of the integrated energy system 560.

In some aspects of the present technology, the integrated energy system 560 can be highly efficient and produce little or no carbon emissions. In additional aspects of the present technology, the processes do not produce or discharge environmentally damaging chemicals.

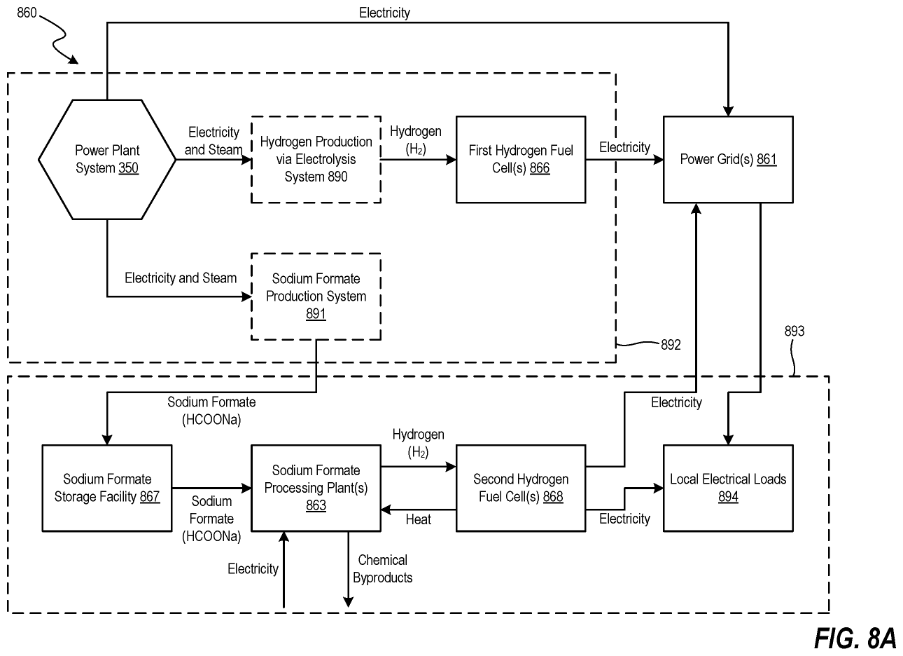

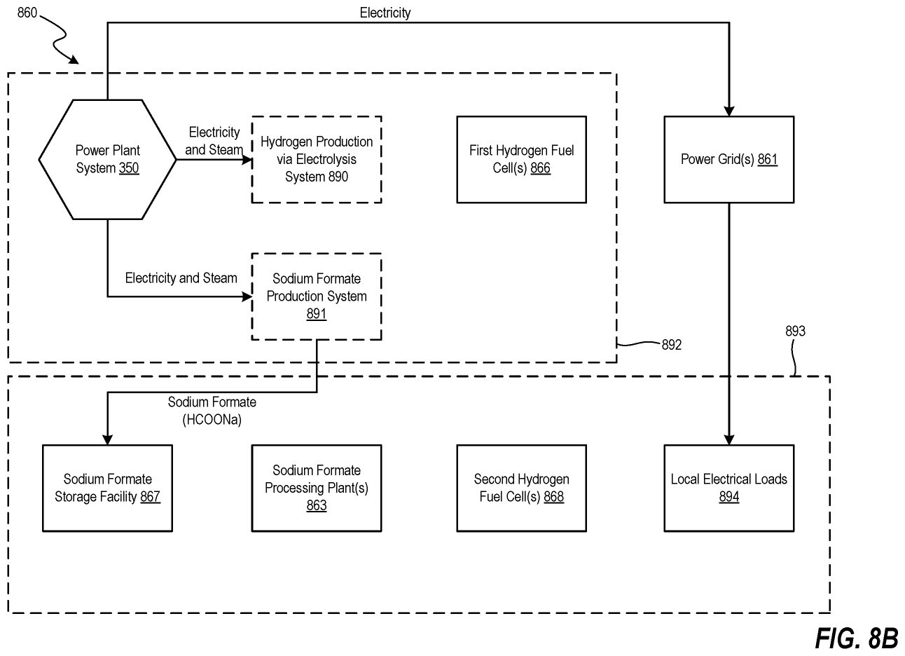

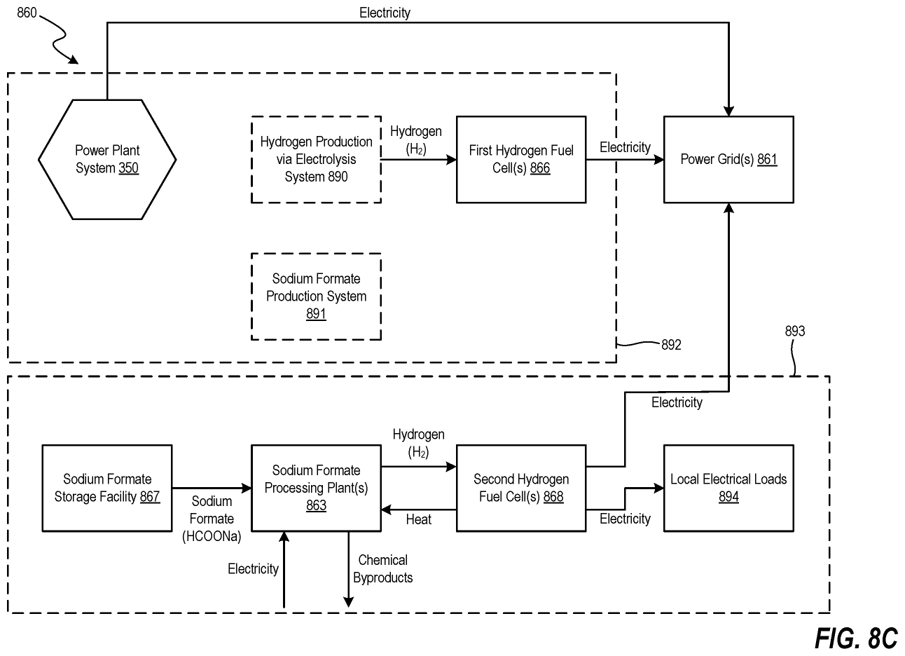

FIG. 8A is a schematic diagram of an integrated energy system 860 including the power plant system 350 of FIG. 3 in accordance with additional embodiments of the present technology. The integrated energy system 860 can include some features that are at least generally similar in structure and function, or identical in structure and function, to the corresponding features of the integrated energy system 460 and/or the integrated energy system 560 described in detail above with reference to FIGS. 4A-5, and can operate in a generally similar or identical manner to the integrated energy system 460 and/or the integrated energy system 560.

For example, in the illustrated embodiment the power plant system 350 is configured to generate electricity and steam and route the electricity and steam (e.g., via one or more power lines, via the electrical power transmission system 354 of FIG. 3, via one or more steam transmission lines, via the steam transmission system 356 of FIG. 3) to (i) one or more components, plants, etc., for producing hydrogen via electrolysis (“hydrogen production via electrolysis system 890”) and (ii) one or more components, plants, etc., for producing sodium formate (“sodium formate production system 891”). The power plant system 350 can further generate electricity and route the electricity to a power rid 861. Referring to FIGS. 4A and 8A, the hydrogen production via electrolysis system 890 can include the hydrogen and oxygen production plant 462, the hydrogen storage facility 464, and the oxygen storage facility 465 described in detail above with reference to FIGS. 4A-4C and can operate in a similar or identical manner to produce hydrogen. The produced hydrogen can be routed to a first hydrogen fuel cell 866 for regenerating electricity for consumption by the power grid 861, such as during peak periods of demand. Referring to FIGS. 5 and 8A, the sodium formate production system 891 can include the desalination plant 571, the brine processing plant 574, the carbon monoxide production plant 573, and the sodium formate production plant 572 described in detail above with reference to FIG. 5 and can operate in a similar or identical manner to produce sodium formate.

Referring to FIG. 8A, in some embodiments each of the power plant system 350, the hydrogen production via electrolysis system 890, and the sodium formate production system 891 can be positioned at a first site 892 local to one another. The first site 892 can comprise a geographic location or area, such as a marine location with a supply of seawater or brackish water for desalination via the sodium formate production system 891. The various components can be located within 0.4 km (0.25 mile), within 0.8 km (0.5 mile), within 3.22 km (2 miles), within 4.82 km (3 miles), or within 8.1 km (5 miles) of one another. In some embodiments, the power plant system 350 includes four, six, twelve, or a different number of the nuclear reactors 300 (FIG. 3) and has a power output of between 300-1000 megawatts electrical (MWe). In some embodiments, the power plant system 350 can output between about 200-600 MWe and between about 1000-3000 megawatts thermal (MWt).

In the illustrated embodiment, the produced sodium formate can be shipped to a second site 893 remote from (e.g., off-site, non-local) from the first site 892. In the illustrated embodiment, the integrated energy system 860 further comprises, at the second site 893, one or more sodium formate storage facilities 867, one or more sodium formate processing plants 863, one or more second hydrogen fuel cells 868, and one or more local electrical loads 894. In some embodiments, the local electrical loads 894 are powered by the power grid 861 and/or another power source during normal operation. The local electrical loads 894 can comprise one or more industrial processing plants, direct air capture plants, and/or the like. The sodium formate processing plant 863 can operate similarly or identically to the sodium formate processing plant 463 and/or the sodium formate processing plant 563 described in detail above with reference to FIGS. 4A-5 and utilize electricity and/or steam from the power plant system 350, the power grid 861, and/or another source to process the sodium formate from the sodium formate storage facility 867 to produce hydrogen and other chemical byproducts such as sodium carbonate (according to equation (2) above), sodium bicarbonate (according to equation (4) above), sodium oxalate, and/or other chemicals. The resultant hydrogen produced by the sodium formate processing plant 863 can be routed (e.g., as an energy carrier) to the second hydrogen fuel cell 868 and used thereby to generate electricity as described in detail above with reference to FIGS. 4A-4C. More specifically, the electricity generated from the hydrogen by the second hydrogen fuel cell 868 can be routed to the local electrical loads 894 at the second site 893 and/or to the power grid 861.

In some embodiments, heat generated from the second hydrogen fuel cell 868 can be routed to the sodium formate processing plant 863 for use in processing the sodium formate to produce hydrogen. For example, heat generated from the electricity can be used upon initial startup of the sodium formate processing plant 863 and then heat generated from the second hydrogen fuel cell 868 can be used to at least partially sustain the sodium formate reaction during operation.

In some aspects of the present technology, the sodium formate produced by the sodium formate production system 891 is a salt that can be easily and safely transported from the first site 892 to the second site 893 and stored at the second site 893. For example, the sodium formate can be transported via truck, tractor, rail, and/or the like from the first site 892 to the second site 893. The second site 893 can be located more than 8.1 km (5 miles), more than 15 km (9.3 miles), more than 30 km (18.6 miles), more than 50 km (31.1 miles), more than 100 km (62.1 miles), more than 500 km (310.7 miles), or farther from the first site.

In some embodiments, the power plant system 350 can be controlled to selectively provide differing amounts of electricity and/or steam to (i) the power grid 861, (ii) the hydrogen production via electrolysis system 890, and (iii) the sodium formate production system 891 based on the demands of the power grid 861 to support an energy imbalance market (EIM). For example, FIGS. 8B and 8C are schematic diagrams of the integrated energy system 860 configured during off-peak hours (e.g., a first demand state of the power grid 861) and during peak hours (e.g., typically during the 4-hour period between 6 pm to 10 pm; e.g., a second demand state of the power grid 861 greater than the first demand state of the power grid 861), respectively, in accordance with embodiments of the present technology.

Referring to FIG. 8B, during off-peak hours, the power plant system 350 can be controlled to have a first operating state in which the power plant system 350 provides excess electricity and steam (i) to the hydrogen production via electrolysis system 890 to generate hydrogen (as an energy carrier) and oxygen for short term storage and/or (ii) to provide excess electricity and steam to the sodium formate production system 891 to produce sodium formate. The sodium formate can subsequently be transported from the first site 892 to the second site 893 and stored at the sodium formate storage facility 867.

Referring to FIG. 8C, during peak hours or other periods of excess or unexpected demand, the power plant system 350 can be controlled to have a second operating state different than the first operating state in which more (e.g., all) of the electricity can be routed from the power plant system 350 to the power grid 861. Additionally, to generate additional electricity for the power grid 861, the first hydrogen fuel cell 866 can be operated at the first site 892 to produce additional electricity from the hydrogen produced during the off-peak hours. Similarly, the sodium formate processing plant 863 can be operated at the second site 893 to generate hydrogen and the second hydrogen fuel cell 868 can generate electricity from the hydrogen for routing to the power grid 861 and/or to the local electrical loads 894, thereby reducing the demand on the power grid 861.

Accordingly, referring to FIGS. 8A-8C, the integrated energy system 860 can (i) route excess steam and electricity from the power plant system 350 to the hydrogen production via electrolysis system 890 to produce hydrogen and to the sodium formate production system 891 to produce sodium formate during off-peak hours when the demands of the power grid 861 are less and then (ii) utilize the produced hydrogen as well as hydrogen produced in-situ at the second site 893 from the sodium formate via the sodium formate processing plant 863 to regenerate electricity via the first hydrogen fuel cell 866 and the second hydrogen fuel cell 868 to meet the greater demands of the power grid 861 during peak hours and/or other unexpected sudden requirements.

In some aspects of the present technology, some of nuclear reactors 300 (FIG. 3) of the power plant system 350 can be dynamically switched from producing electricity for routing to the power grid 861 to producing steam and/or electricity for use in the hydrogen production via electrolysis system 890 to produce hydrogen and the sodium formate production system 891 to produce sodium formate. Accordingly, the modularity of the nuclear reactors 300 allows the power plant system 350 to flexibly/dynamically switch the output of electricity and steam from individual ones of the nuclear reactors 300 based on the demands of the integrated energy system 860 (e.g., the power grid 861).

Additionally, one or more of the nuclear reactors 300 can be individually taken offline for servicing, maintenance, refueling, etc., while the remainder of the nuclear reactors 300 can continue to produce steam and/or electricity. Accordingly, the power plant system 350 can continue to provide steam and electricity to the power grid 861, the hydrogen production via electrolysis system 890, and/or the sodium formate production system 891, even during servicing, maintenance, refueling, etc. In contrast, conventional nuclear reactor systems must be entirely shut down during such procedures such that neither steam nor electricity are available.

In some aspects of the present technology, the integrated energy system 860 can be highly efficient and produce little or no carbon emissions. In contrast, conventional systems for producing hydrogen and/or sodium formate generally rely on steam-methane reforming which reacts natural gas with steam at elevated temperature to produce carbon monoxide and hydrogen. Steam-methane reforming has significant carbon emissions—generally producing about 9.3 kilograms (kg) of carbon dioxide per kg of hydrogen produced.

Referring to FIGS. 4A-8C, the various components can be combined and/or omitted to form integrated energy systems having different configurations. For example, an integrated energy system in accordance with the present technology can be configured to selectively supply electricity to a power grid and electricity and/or steam to a sodium formate production process without routing electricity and steam to a hydrogen production via electrolysis process. That is, for example, the hydrogen production via electrolysis system 890 can be omitted from the integrated energy system 860 shown in FIG. 8.

Referring to FIGS. 4A-8C, each of the arrows indicating the routing/transfer of steam, electricity, sodium formate, hydrogen, oxygen, sodium hydroxide, chlorine, other chemical products, etc., can indicate a portion of the routing of overall production of each component. For example, referring to FIG. 4A, the power plant system 350 can route a first portion of the electricity generated by the power plant system 350 to the power grid 461, a second portion of the electricity to the hydrogen and oxygen production plant 462, a third portion of the electricity to the sodium formate processing plant 463, and so on. Similarly, with reference to FIG. 5, the integrated energy system 560 can route a first portion of the hydrogen produced by the brine processing plant 574 and/or the sodium formate processing plant 563 to the hydrochloric acid production plant 577 and a second portion of the hydrogen to other uses as an energy carrier.

Moreover, while reference is typically made herein to generating “steam,” the power plant system 350 can be used to produce other gases. For example, other fluids can be fed into the nuclear reactors 300 (FIG. 3) and heated to produce gases other than steam that can be routed to various components of the integrated energy systems.

### III. Additional Examples

The following examples are illustrative of several embodiments of the present technology:
1. An integrated energy system, comprising:
a power grid, wherein the power grid has a first demand state and a second demand state greater than the first demand state;
a sodium formate production system, wherein the sodium formate production system is configured to produce sodium formate; and
a power plant system operably coupled to the power grid and the sodium formate production system, wherein the power plant system includes a plurality of nuclear reactors and an electrical power conversion system, wherein individual ones of the nuclear reactors are configured to heat a coolant into steam, and wherein—
   during the first demand state of the power grid, the power plant system is configured to have a first operating state in which (a) the steam from a first subset of the nuclear reactors is routed to the electrical power conversion system to generate electricity that is routed to the power grid and (b) the steam from a second subset of the nuclear reactors is routed to the sodium formate production system for use in producing the sodium formate and/or to the electrical power conversion system to generate electricity that is routed to the sodium formate production system for use in producing the sodium formate; and
   during the second demand state of the power grid, the power plant system is configured to have a second operating state different from the first operating state in which the steam from at least one of the nuclear reactors in the second subset is routed to the electrical power conversion system to generate electricity that is routed to the power grid.
2. The integrated energy system of example 1, further comprising a sodium formate processing plant configured to receive the sodium formate produced by the sodium formate production system and process the sodium formate to produce hydrogen.
3. The integrated energy system of example 2 wherein the sodium formate production system and the power plant system are positioned local to one another at a first site, and wherein the sodium formate processing plant is positioned at a second site remote from the first site.
4. The integrated energy system of example 3, further comprising a hydrogen fuel cell positioned at the second site to receive the hydrogen produced by the sodium formate processing plant and configured to process the hydrogen to generate electricity.
5. The integrated energy system of example 4 wherein the hydrogen fuel cell is operably coupled to the power grid, and wherein, during the second demand state of the power grid—
the sodium formate processing plant is configured to process the sodium formate to produce the hydrogen; and
the hydrogen fuel cell is configured to process the hydrogen to generate the electricity and route the electricity to the power grid.
6. The integrated energy system of example 4 or example 5 wherein, during the second demand state of the power grid—
the sodium formate processing plant is configured to process the sodium formate to produce the hydrogen; and
the hydrogen fuel cell is configured to process the hydrogen to generate the electricity and route the electricity to at least one electrical load positioned at the second site.
7. The integrated energy system of any one of examples 1-6 wherein the sodium formate production system comprises:
a desalination plant positioned to receive seawater or brackish water, wherein the desalination plant is operably coupled to the power plant system, and wherein, during the first demand state of the power grid, the desalination plant is configured receive the steam from the second subset of the nuclear reactors and/or the electricity from the second subset of the nuclear reactors and use the steam and/or the electricity to process the seawater or brackish water to produce brine and clean water;
a brine processing plant positioned to receive the brine from the desalination plant and configured to process the brine to produce sodium hydroxide; and
a sodium formate production plant positioned to receive the sodium hydroxide from the brine processing plant and carbon monoxide from a carbon monoxide source and configured to process the sodium hydroxide and the carbon monoxide to produce the sodium formate.
8. The integrated energy system of example 7 wherein the desalination plant is positioned at a coastal location and/or near a location of brackish water.
9. The integrated energy system of any one of examples 1-8 wherein the power plant system is floating.
10. The integrated energy system of any one of examples 1-9, further comprising an electrolysis system operably coupled to the power plant, wherein—
in the first operating state of the power plant system, the steam from a third subset of the nuclear reactors is routed to the electrolysis system and/or to the electrical power conversion system to generate electricity that is routed to the electrolysis system; and the electrolysis system is configured to utilize the steam and/or electricity from the third subset of the nuclear reactors in an electrolysis process to produce hydrogen.
11. The integrated energy system of example 10 wherein the electrolysis process is a high temperature steam electrolysis process.
12. The integrated energy system of example 10 or example 11 wherein, in the second operating state of the power plant system, the steam from at least one of the nuclear reactors in the third subset is routed to the electrical power conversion system to generate electricity that is routed to the power grid.
13. The integrated energy system of any one of examples 10-12, further comprising a hydrogen fuel cell positioned to receive the hydrogen produced by the electrolysis system, and wherein, during the second demand state of the power grid, the hydrogen fuel cell is configured to process the hydrogen to generate electricity and route the electricity to the power grid.
14. An integrated energy system, comprising:
a power plant system including a plurality of nuclear reactors, wherein the power plant system is configured to produce a steam output and an electrical output;
a desalination plant positioned to receive seawater or brackish water and operably coupled to the power plant system, wherein the desalination plant is configured to receive a first portion of the steam output and/or a first portion of the electrical output and use the first portion of the steam output and/or the first portion of the electrical output to process the seawater or brackish water to produce brine and clean water;
a brine processing plant operably coupled to the desalination plant and configured to receive the brine from the desalination plant and to process the brine to produce sodium hydroxide; and
a sodium formate production plant positioned to receive the sodium hydroxide from the brine processing plant and carbon monoxide from a carbon monoxide source, wherein the sodium formate production plant is operably coupled to the power plant system and configured to receive a second portion of the steam output and/or a second portion of the electrical output and use the second portion of the steam output and/or the second portion of the electrical output to process the sodium hydroxide and the carbon monoxide to produce sodium formate.
15. The integrated energy system of example 14 wherein the brine processing plant is configured to treat the brine using a chlor-alkali membrane electrolysis process to produce the sodium hydroxide.
16. The integrated energy system of example 14 or example 15, further comprising the carbon monoxide source, wherein the carbon monoxide source comprises a carbon monoxide production plant, and wherein the carbon monoxide production plant is operably coupled to the power plant system and configured to receive a third portion of the steam output and/or a third portion of the electrical output and use the third portion of the steam output and/or the third portion of the electrical output to process carbon dioxide to produce the carbon monoxide.
17. The integrated energy system of any one of examples 14-16 wherein the sodium formate production plant comprises a reaction chamber positioned to receive the carbon monoxide and the sodium hydroxide, and wherein the sodium formate production plant is configured to utilize the second portion of the steam output and/or the second portion of the electrical output to heat the reaction chamber to about 130° C. and to pressurize the reaction chamber to between about 6-8 bar such that the carbon monoxide is absorbed into the sodium hydroxide to produce the sodium formate.
18. The integrated energy system of any one of examples 14-17 wherein the brine processing plant is further configured to process the brine to produce hydrogen and chlorine, and further comprising a hydrochloric acid production plant positioned to receive the hydrogen and chlorine from the brine processing plant and the clean water from the desalination plant and to process the hydrogen, the chlorine, and the clean water to produce hydrochloric acid.
19. The integrated energy system of any one of examples 14-18 wherein the power plant system and the desalination plant are positioned local to one another.
20. A method of operating an integrated energy system for supplying electricity to a power grid, wherein the integrated energy system includes a power plant having a plurality of nuclear reactors and an electrical power conversion system, and wherein individual ones of the nuclear reactors are configured to heat a coolant into steam, the method comprising:
during a first demand state of the power grid, configuring the power plant system in a first operating state in which (a) the steam from a first subset of the nuclear reactors is routed to the electrical power conversion system to generate electricity that is routed to the power grid and (b) the steam from a second subset of the nuclear reactors is routed to a sodium formate production system for use in producing sodium formate and/or to the electrical power conversion system to generate electricity that is routed to the sodium formate production system for use in producing the sodium formate; and
during a second demand state of the power grid greater than the first demand state, configuring the power plant system in a second operating state different from the first operating state in which the steam from at least one of the nuclear reactors in the second subset is routed to the electrical power conversion system to generate electricity that is routed to the power grid.

### IV. CONCLUSION

All numeric values are herein assumed to be modified by the term about whether or not explicitly indicated. The term about, in the context of numeric values, generally refers to a range of numbers that one of skill in the art would consider equivalent to the recited value (e.g., having the same function and/or result). For example, the term about can refer to the stated value plus or minus ten percent. For example, the use of the term about 100 can refer to a range of from 90 to 110, inclusive. In instances in which the context requires otherwise and/or relative terminology is used in reference to something that does not include, or is not related to, a numerical value, the terms are given their ordinary meaning to one skilled in the art.

The above detailed description of embodiments of the present technology are not intended to be exhaustive or to limit the technology to the precise forms disclosed above. Although specific embodiments of, and examples for, the technology are described above for illustrative purposes, various equivalent modifications are possible within the scope of the technology, as those skilled in the relevant art will recognize. For example, although steps may be presented in a given order, in other embodiments, the steps may be performed in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

As used herein, the phrase and/or as in A and/or B refers to A alone, B alone, and A and B. Additionally, the term comprising is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An integrated energy system, comprising:
a power grid, wherein the power grid has a first demand state and a second demand state greater than the first demand state;
a sodium formate production system, wherein the sodium formate production system is configured to produce sodium formate; and
a power plant system operably coupled to the power grid and the sodium formate production system, wherein the power plant system includes a plurality of nuclear reactors and an electrical power conversion system, wherein individual ones of the nuclear reactors are configured to heat a coolant into steam, and wherein—
during the first demand state of the power grid, the power plant system is configured to have a first operating state in which (a) the steam from a first subset of the nuclear reactors is routed to the electrical power conversion system to generate electricity that is routed to the power grid and (b) the steam from a second subset of the nuclear reactors is routed to the sodium formate production system for use in producing the sodium formate and/or to the electrical power conversion system to generate electricity that is routed to the sodium formate production system for use in producing the sodium formate; and
during the second demand state of the power grid, the power plant system is configured to have a second operating state different from the first operating state in which the steam from at least one of the nuclear reactors in the second subset is routed to the electrical power conversion system to generate electricity that is routed to the power grid.

2. The integrated energy system of claim 1, further comprising a sodium formate processing plant configured to receive the sodium formate produced by the sodium formate production system and process the sodium formate to produce hydrogen.

3. The integrated energy system of claim 2 wherein the sodium formate production system and the power plant system are positioned local to one another at a first site, and wherein the sodium formate processing plant is positioned at a second site remote from the first site.

4. The integrated energy system of claim 3, further comprising a hydrogen fuel cell positioned at the second site to receive the hydrogen produced by the sodium formate processing plant and configured to process the hydrogen to generate electricity.

5. The integrated energy system of claim 4 wherein the hydrogen fuel cell is operably coupled to the power grid, and wherein, during the second demand state of the power grid—
the sodium formate processing plant is configured to process the sodium formate to produce the hydrogen; and
the hydrogen fuel cell is configured to process the hydrogen to generate the electricity and route the electricity to the power grid.

6. The integrated energy system of claim 4 wherein, during the second demand state of the power grid—
the sodium formate processing plant is configured to process the sodium formate to produce the hydrogen; and
the hydrogen fuel cell is configured to process the hydrogen to generate the electricity and route the electricity to at least one electrical load positioned at the second site.

7. The integrated energy system of claim 1 wherein the sodium formate production system comprises:
a desalination plant positioned to receive seawater or brackish water, wherein the desalination plant is operably coupled to the power plant system, and wherein, during the first demand state of the power grid, the desalination plant is configured receive the steam from the second subset of the nuclear reactors and/or the electricity from the second subset of the nuclear reactors and use the steam and/or the electricity to process the seawater or brackish water to produce brine and clean water;
a brine processing plant positioned to receive the brine from the desalination plant and configured to process the brine to produce sodium hydroxide; and
a sodium formate production plant positioned to receive the sodium hydroxide from the brine processing plant and carbon monoxide from a carbon monoxide source and configured to process the sodium hydroxide and the carbon monoxide to produce the sodium formate.

8. The integrated energy system of claim 7 wherein the desalination plant is positioned at a coastal location and/or near a location of brackish water.

9. The integrated energy system of claim 1 wherein the power plant system is floating.

10. The integrated energy system of claim 1, further comprising an electrolysis system operably coupled to the power plant, wherein—
in the first operating state of the power plant system, the steam from a third subset of the nuclear reactors is routed to the electrolysis system and/or to the electrical power conversion system to generate electricity that is routed to the electrolysis system; and
the electrolysis system is configured to utilize the steam and/or electricity from the third subset of the nuclear reactors in an electrolysis process to produce hydrogen.

11. The integrated energy system of claim 10 wherein the electrolysis process is a high temperature steam electrolysis process.

12. The integrated energy system of claim 10 wherein, in the second operating state of the power plant system, the steam from at least one of the nuclear reactors in the third subset is routed to the electrical power conversion system to generate electricity that is routed to the power grid.

13. The integrated energy system of claim 10, further comprising a hydrogen fuel cell positioned to receive the hydrogen produced by the electrolysis system, and wherein, during the second demand state of the power grid, the hydrogen fuel cell is configured to process the hydrogen to generate electricity and route the electricity to the power grid.

14. An integrated energy system, comprising:
a power plant system including a plurality of nuclear reactors, wherein the power plant system is configured to produce a steam output and an electrical output;
a desalination plant positioned to receive seawater or brackish water and operably coupled to the power plant system, wherein the desalination plant is configured to receive a first portion of the steam output and/or a first portion of the electrical output and use the first portion of the steam output and/or the first portion of the electrical output to process the seawater or brackish water to produce brine and clean water;
a brine processing plant operably coupled to the desalination plant and configured to receive the brine from the desalination plant and to process the brine to produce sodium hydroxide; and
a sodium formate production plant positioned to receive the sodium hydroxide from the brine processing plant and carbon monoxide from a carbon monoxide source, wherein the sodium formate production plant is operably coupled to the power plant system and configured to receive a second portion of the steam output and/or a second portion of the electrical output and use the second portion of the steam output and/or the second portion of the electrical output to process the sodium hydroxide and the carbon monoxide to produce sodium formate.

15. The integrated energy system of claim 14 wherein the brine processing plant is configured to treat the brine using a chlor-alkali membrane electrolysis process to produce the sodium hydroxide.

16. The integrated energy system of claim 14, further comprising the carbon monoxide source, wherein the carbon monoxide source comprises a carbon monoxide production plant, and wherein the carbon monoxide production plant is operably coupled to the power plant system and configured to receive a third portion of the steam output and/or a third portion of the electrical output and use the third portion of the steam output and/or the third portion of the electrical output to process carbon dioxide to produce the carbon monoxide.

17. The integrated energy system of claim 14 wherein the sodium formate production plant comprises a reaction chamber positioned to receive the carbon monoxide and the sodium hydroxide, and wherein the sodium formate production plant is configured to utilize the second portion of the steam output and/or the second portion of the electrical output to heat the reaction chamber to about 130° C. and to pressurize the reaction chamber to between about 6-8 bar such that the carbon monoxide is absorbed into the sodium hydroxide to produce the sodium formate.

18. The integrated energy system of claim 14 wherein the brine processing plant is further configured to process the brine to produce hydrogen and chlorine, and further comprising a hydrochloric acid production plant positioned to receive the hydrogen and chlorine from the brine processing plant and the clean water from the desalination plant and to process the hydrogen, the chlorine, and the clean water to produce hydrochloric acid.

19. The integrated energy system of claim 14 wherein the power plant system and the desalination plant are positioned local to one another.

20. A method of operating an integrated energy system for supplying electricity to a power grid, wherein the integrated energy system includes a power plant having a plurality of nuclear reactors and an electrical power conversion system, and wherein individual ones of the nuclear reactors are configured to heat a coolant into steam, the method comprising:
during a first demand state of the power grid, configuring the power plant system in a first operating state in which (a) the steam from a first subset of the nuclear reactors is routed to the electrical power conversion system to generate electricity that is routed to the power grid and (b) the steam from a second subset of the nuclear reactors is routed to a sodium formate production system for use in producing sodium formate and/or to the electrical power conversion system to generate electricity that is routed to the sodium formate production system for use in producing the sodium formate; and
during a second demand state of the power grid greater than the first demand state, configuring the power plant system in a second operating state different from the first operating state in which the steam from at least one of the nuclear reactors in the second subset is routed to the electrical power conversion system to generate electricity that is routed to the power grid.
